(19) United States
(12) Patent Application Publication
(10) Pub. No.: US 2010/0267731 A1
(43) Pub. Date: Oct. 21, 2010

(54) USE OF TRANSFORMING GROWTH FACTOR-B RECEPTOR INHIBITORS TO SUPPRESS OCULAR SCARRING

(75) Inventor: Hiroshi Nakamura, Hudson, OH (US)

Correspondence Address: BENESCH, FRIEDLANDER, COPLAN & ARONOFF LLP;ATTN: IP DEPARTMENT DOCKET CLERK, 200 PUBLIC SQUARE, SUITE 2300, CLEVELAND, OH, 44114-2378, US

(73) Assignee: Summa Health Systems, LLC, Akron, OH (US)

(21) Appl. No.: 12/761,982

(22) Filed: Apr. 16, 2010

Related U.S. Application Data
(60) Provisional application No. 61/170,141, filed on Apr. 17, 2009.

(51) Int. Cl. A61K 31/498; A61K 31/4439; A61K 31/4375; A61K 31/4418; A61K 31/4709; A61K 31/444; A61P 27/02

(52) U.S. Cl. 514/249; 514/338; 514/300; 514/333; 514/314; 514/335

(57) ABSTRACT

A pharmaceutical composition useful in the prevention of subconjunctival scarring that may occur after GFS comprising an effective amount of an ALK-5 inhibitor. Also disclosed is a method of treating disorder or condition of other ocular scarring or fibrosis including corneal haze and PVR that may develop after ocular surgery or injury comprising applying an amount of a pharmaceutical composition including an ALK-5 inhibitor to a post-surgical or injury site.

This application claims priority to U.S. Provisional Patent Application No. 61/170,141, dated Apr. 17, 2009.

The development of this invention was supported by funding from the American Health Assistance Foundation, G2006-014. The government has an interest in the invention.

### BACKGROUND

Ocular fibrotic wound response is a major cause of impaired vision and blindness, especially as a consequence of the surgical treatment for glaucoma. Glaucoma is a leading cause of blindness in the United States, and 2.5 million Americans and 65 million people worldwide were affected by the disease in 2000. Glaucoma is a disease characterized by damage to the optic nerve head, and neural and visual loss. One of major risk factors of glaucoma is an elevated intraocular pressure (IOP) resulting from abnormalities in the aqueous humor outflow pathway. Glaucoma filtration surgery (GFS) is commonly performed when medication fails to control IOP adequately.

Excessive post-operation scarring often leads to failure of GFS. While the use of antimetabolites such as mitomycin-C (MMC) and 5-fluorouracil as conjunctival anti-scarring treatments benefits a number of patients, they do so by causing widespread cell death and are associated with severe and potentially blinding complications, such as hypotony maculopathy and infection. Therefore, other anti-scarring approaches have been investigated. In particular, transforming growth factor beta (TGF-β) and its pathway have emerged as a target for postoperative anti-scarring therapy.

### BRIEF DESCRIPTION OF DRAWINGS

The accompanying drawings, which are incorporated in and constitute a part of the specification, illustrate various example systems, methods, and so on, that illustrate various example embodiments of aspects of the invention. It will be appreciated that the illustrated element boundaries (e.g., boxes, groups of boxes, or other shapes) in the figures represent one example of the boundaries. One of ordinary skill in the art will appreciate that one element may be designed as multiple elements or that multiple elements may be designed as one element. An element shown as an internal component of another element may be implemented as an external component and vice versa. Furthermore, elements may not be drawn to scale.

FIG. 1 a side view of a human eye during GFS.

FIG. 2 is a graph showing the effect of ALK-5 inhibitor 616451 on the TGF-β signaling levels in cultured rabbit subconjunctival fibroblasts.

FIG. 3 is a graph showing the effect of ALK-5 inhibitor SB-505124 on the TGF-β signaling levels in cultured rabbit subconjunctival fibroblasts.

FIG. 4 is a graph showing the effect of ALK-5 inhibitor 606452 on the TGF-β signaling levels in cultured rabbit subconjunctival fibroblasts.

FIG. 5 is a graph showing the effect of ALK-5 inhibitor SD-208 on the TGF-β signaling levels in cultured rabbit subconjunctival fibroblasts.

FIG. 6 is a graph showing the effect of ALK-5 inhibitor SB-525334 on the TGF-β signaling levels in cultured rabbit subconjunctival fibroblasts.

FIG. 7 is a graph showing the cell toxicity of ALK-5 inhibitor SB-505124 on cultured rabbit subconjunctival fibroblasts.

FIG. 8 is a graph showing the effect of ALK-5 inhibitor SB-505124 and various controls on animal subjects.

FIG. 9A is a graph showing the effect of ALK-5 inhibitor SB-505124 on the IOP of animal subjects.

FIG. 9B is a graph showing the effect of mitomytocin-c on the IOP of animal subjects.

FIG. 9C is a graph showing the effect of no treatment on the IOP of animal subjects.

FIG. 9D is a graph showing the effect of a lactose control tablet on the IOP of animal subjects.

FIGS. 10A-C are images showing the effect of ALK-5 inhibitor SB-505124 and various controls on the eves of animal subjects.

FIG. 11A-C are images showing the effect of ALK-5 inhibitor SB-505124 and various controls on the cell outgrowth of an explant taken from the eyes of animal subjects.

FIGS. 12A-D are graphs showing the effect of ALK-5 inhibitor SB-505124 and various controls on the cell outgrowth from explants taken from the eyes of animal subjects.

### DETAILED DESCRIPTION

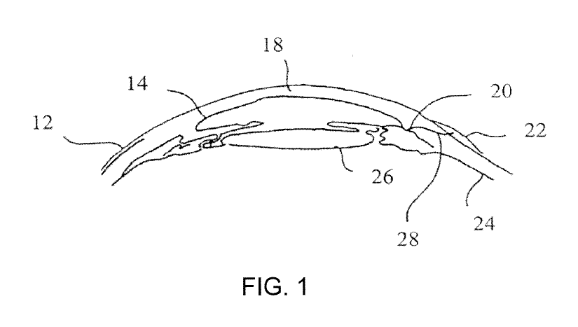

Disclosed herein are methods for preventing and treating ocular scarring following GFS in a mammal. Preferably, the method may be used to treat human patients during or following GFS. In GFS, a new drainage site is created to facilitate drainage of fluid from the eye, thereby decreasing the IOP in the eye. As shown in FIG. 1, the human eye includes the conjunctiva 12, trabecular meshwork 14, iris 16, cornea 18, retina 24, and lens 26, among other components.

During GFS, instead of draining into the normal drainage site (the trabecular meshwork) 14 of the eye, the aqueous humor is drained into a new “space” that is created under the conjunctiva 12 of the eye. To do this, a small flap in the white of the eye is made. This is followed by the creation of a new drainage route 28 between the opening of the route 20 and a reservoir called a filtration bleb 22. The fluid in the anterior and posterior chamber, called the aqueous humor, can then drain into the bleb 22 via the new drainage route 28 and be absorbed into the vessels around the eve. The bleb 22 and/or the new drainage route 28 can scar and close preventing the aqueous humor from properly draining, called bleb failure.

TGF-β is a key mediator of wound healing responses. In the eye. TGF-β has been implicated in causing corneal haze after corneal injury and laser surgery and subconjunctival scarring following GFS. In addition, TGF-β upregulation is involved in proliferative vitreoretinopathy (PVR), which is a major cause for the failure of retinal detachment surgery.

The activin receptor-like kinase (ALK) 5 inhibitors have been identified to block the TGF-β signaling pathway, and thus, may be used to prevent corneal haze after corneal injury and laser surgery such as LASIK, and scarring following ocular surgery, including GFS, and corneal and vitreo-retinal surgeries. Also, the use of the ALK-5 inhibitors may reduce the side effects, including late onset post surgical infection associated with tissue damage caused by current anti-scarring reagent, such as MMC. Other side effects may include bleeding, swelling, scarring, retinal detachment, a droopy eyelid, double vision, loss of vision, or even loss of the eye. Finally, topical application of ALK-5 inhibitors to the human eye may lower the IOP associated with glaucoma.

In one embodiment, a method to suppress ocular scaring following a GFS procedure or ocular injury includes providing a composition comprising an effective amount of an ALK-5 inhibitor, in an amount sufficient to inhibit the signaling pathway of TGF-β, and a pharmaceutically acceptable vehicle therefore, and combinations thereof, wherein the application of the composition to a post-surgical or injury site suppresses formation of scar tissue following ocular surgery and/or ocular injury.

One of skill in the art would understand that the formation of the filtering bleb 22, as shown in FIG. 1, is important to the GFS procedure. If the bleb 22 and/or the new drainage route 28 scars or closes, preventing the aqueous humor from properly draining (bleb failure), the filtration surgery may fail. Therefore, the amount of ALK-5 inhibitor used during the surgical procedure should be in an amount sufficient to inhibit the signaling pathway of TGF-β, thus preventing bleb failure. It should be understood that the term “about” means plus or minus 10% of any given number used herein. Preferably, the compositions may include from about 0.3 to about 30 micomolar (μM) of the ALK-5 inhibitor, and more preferably from about 3 to about 15 μM of inhibitor. Moreover, one of skill in the art would understand that compositions including more than 30 μM may also be used.

One or more of the following compounds may be used in a GFS procedure to suppress the formation of scar tissue. Manufacturer designation has been provided where available.

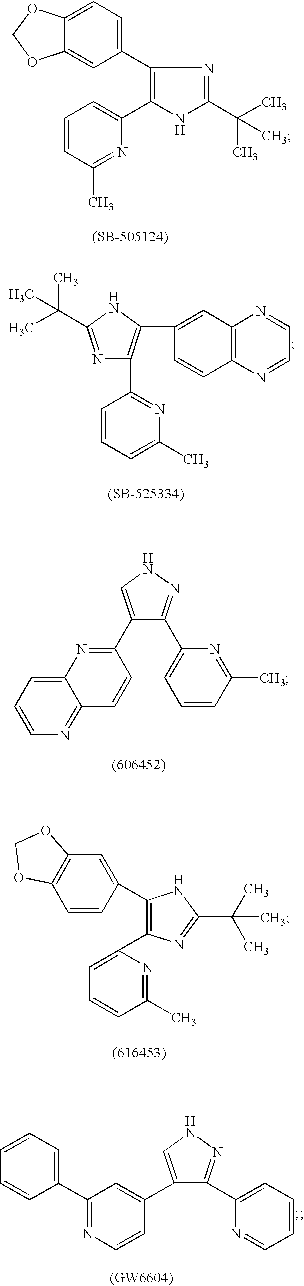

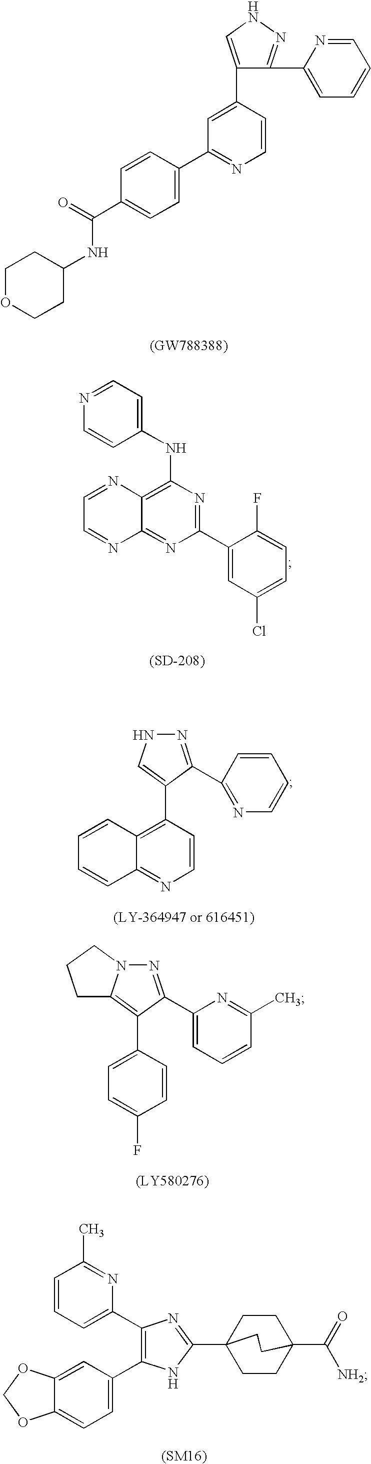

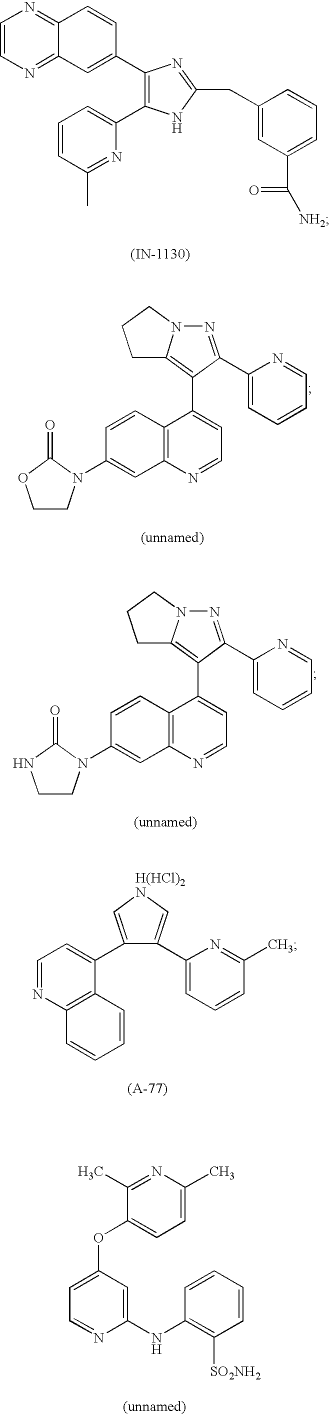

Additional compounds are known by their manufacturer name and include inhibitors KI26894, LY2109761. IN-1233, and SKI2162. From the collection of compounds described above, the following can be obtained from various sources: LY-364947, SB-525334, SD-208, and SB-505124 available from Sigma, P.O. Box 14508, St. Louis, Mo., 63178-9916; 616452 and 616453 available from Calbiochem (EMD Chemicals, Inc.), 480 S. Democrat Road, Gibbstown, N.J., 08027; GW788388 and GW6604 available from GlaxoSmithKline, 980 Great West Road, Brentford, Middlesex, TW8 9GS, United Kingdom; LY580276 available from Lilly Research, Indianapolis, Ind. 46285; and SM16 available from Biogen Idec, P.O. Box 14627, 5000 Davis Drive, Research Triangle Park, N.C., 27709-4627.

The above-described compositions may include ALK-5 inhibitors, and pharmaceutically acceptable salts thereof, that can be combined with various types of pharmaceutically acceptable vehicles to be applied to eyes, such as sustained release polymers, carriers capable of forming gels upon administration, hydrogels, creams, ointments, sprays, liquids, or tablets. The vehicles may be aqueous, and are formulated to be chemically and physically compatible with ophthalmic tissues. For example, bioerodible (or biodegradable) gels or collagen inserts may be used to keep an effective concentration of the inhibitor in the bleb. The use of such gels or inserts has the advantage of providing a sustained release of the active components at the surgical site.

As will be appreciated by those skilled in the art, the above-described compositions should be sterile and should not include any agents which will be toxic to sensitive intraocular tissues, particularly cornea/endothelial cells. The above described compositions can be formulated in accordance with techniques known to those skilled in the art.

The above described ALK-5 inhibitors can be applied to the surgical site by means of various techniques. For example, the compositions can be applied by means of a syringe during or immediately after surgery, preferably within 4 hours, or with a sustained release polymer that can be inserted into the eve on or around the surgical site. The compositions may be applied to the surgical site in a topical formulation following LASIK to prevent or reduce corneal haze.

### EXAMPLES

### Example 1

### Measurement of Suppression of TGF-β2 In Vitro

Sample fibroblasts were obtained from New Zealand white rabbit eyes. The fibroblasts were derived from the subconjunctival tissues isolated from the eves of the subjects. The cells were maintained in 25 cm² flask using 2 ml of medium composed of Eagle's minimal essential medium, 10% fetal bovine serum, 5% calf serum, essential and nonessential aminoacids, and antibiotics. When the cells reached confluence, they were trvpsinized and passaged.

The fibroblast cultures in 6-well plates were pre-treated with 2 ml of medium including ALK-5 inhibitors at various concentrations, 0.03, 0.1, 0.03, 1.0, 3.0, and 10.0 μM, respectively, for one hour, and were additionally treated with additional 2 ng/ml of TGF-β2 (R&D Systems, Minneapolis, Minn.) for up to 48 hours. As shown in Table 1, samples 1-6 were treated with ALK-5 inhibitor 616451, samples 7-12 with ALK-5 inhibitor 616452, samples 13-18 with ALK-5 inhibitor SD-208, samples 19-24 with ALK-5 inhibitor SB-505124, and samples 25-30 with ALK-5 inhibitor SB-525334.

Samples 31 and 32 were prepared as controls. Sample 31 was not treated with an ALK-5 inhibitor or TGF-β2. Sample 32 was treated with 2 ng/ml of TGF-β2, but not with an ALK-5 inhibitor. The samples were prepared as shown in Table 1, below.

TABLE 1

| | | Concentration Inhibitor | |
|---|---|---|---|
| | ALK-5 | (μM) | |
| Sample # | Inhibitor | Pre-treat | TGF-β2 (ng/ml) |
| 1 | 616451 | 0.03 | 2.0 |
| 2 | 616451 | 0.1 | 2,0 |
| 3 | 616451 | 0.3 | 2.0 |
| 4 | 616451 | 1.0 | 2.0 |
| 5 | 616451 | 3.0 | 2.0 |
| 6 | 616451 | 10.0 | 2.0 |
| 7 | 606452 | 0.03 | 2.0 |
| 8 | 606452 | 0.1 | 2.0 |
| 9 | 606452 | 0.3 | 2.0 |
| 10 | 606452 | 1.0 | 2.0 |
| 11 | 606452 | 3.0 | 2.0 |
| 12 | 606452 | 10.0 | 2.0 |
| 13 | SD-208 | 0.03 | 2.0 |
| 14 | SD-208 | 0.1 | 2.0 |
| 15 | SD-208 | 0.3 | 2.0 |
| 16 | SD-208 | 1.0 | 2.0 |
| 17 | SD-208 | 3.0 | 2.0 |
| 18 | SD-208 | 10.0 | 2.0 |
| 19 | SB-505124 | 0.03 | 2.0 |
| 20 | SB-505124 | 0.1 | 2.0 |
| 21 | SB-505124 | 0.3 | 2.0 |
| 22 | SB-505124 | 1.0 | 2.0 |
| 23 | SB-505124 | 3.0 | 2.0 |
| 24 | SB-505124 | 10.0 | 2.0 |
| 25 | SB-525334 | 0.03 | 2.0 |
| 26 | SB-525334 | 0.1 | 2.0 |
| 27 | SB-525334 | 0.3 | 2.0 |
| 28 | SB-525334 | 1.0 | 2.0 |
| 29 | SB-525334 | 3.0 | 2.0 |
| 30 | SB-525334 | 10.0 | 2.0 |
| 31 | N/A | 0.0 | 0.0 |
| 32 | N/A | 0.0 | 2.0 |

Cells from the various samples were harvested following the treatment with the inhibitors and/or TGF-β2, and western blotting was performed to provide quantitative assessments. The conjunctival fibroblasts were lysed in a Triton lysis buffer. The total protein in the lysates was quantified using a Bradford protein assay. Equal amounts of protein (20 μg/lane) were resolved on a 10% sodium dodecyl sulfate (SDS)-polyacrylamide gel. The protein was then transferred to nitrocellulose membranes.

After blocking with 1% bovine serum albumin, the membranes were probed with polyclonal goat anti-connective tissue growth factor (CTGF) (1:200, Santa Cruz Biotechnology, Santa Cruz, Calif.,) followed by HRP-conjugatcd donkey anti-goat IgG (1:1,000; Jackson ImmunoResearch, West Grove, Pa.). The TGF-β signal was detected by enhanced chemiluminescence (ECL) using SuperSignal from Pierce (Rockford, Ill.). Densitometry was then performed to measure the intensity of bands.

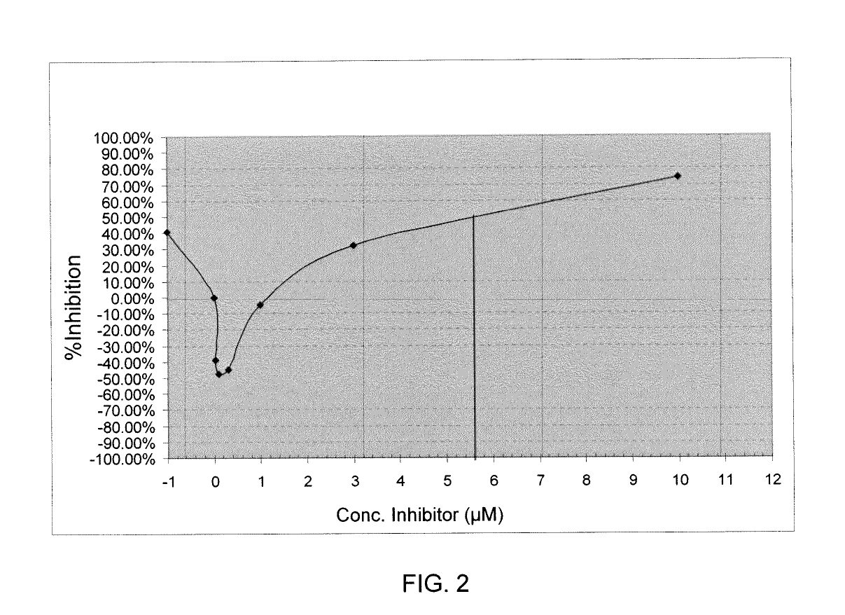

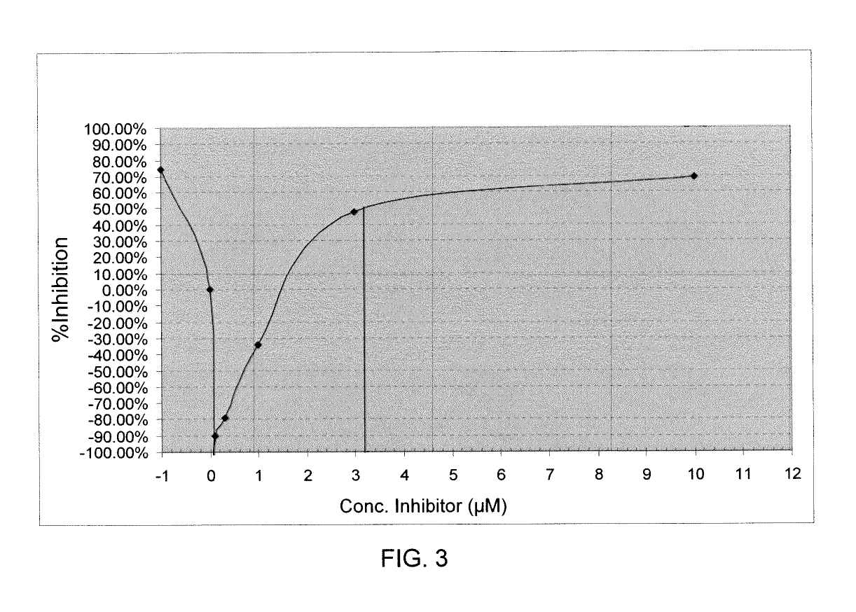

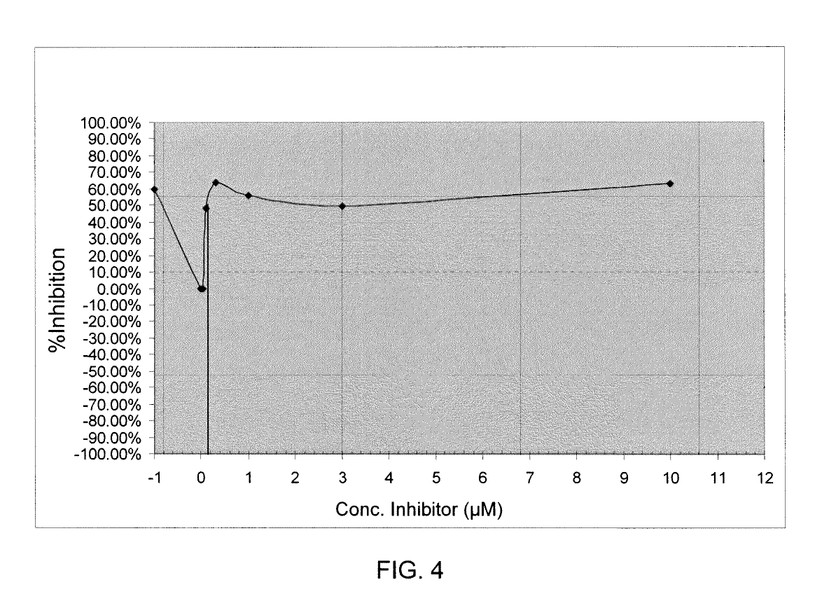

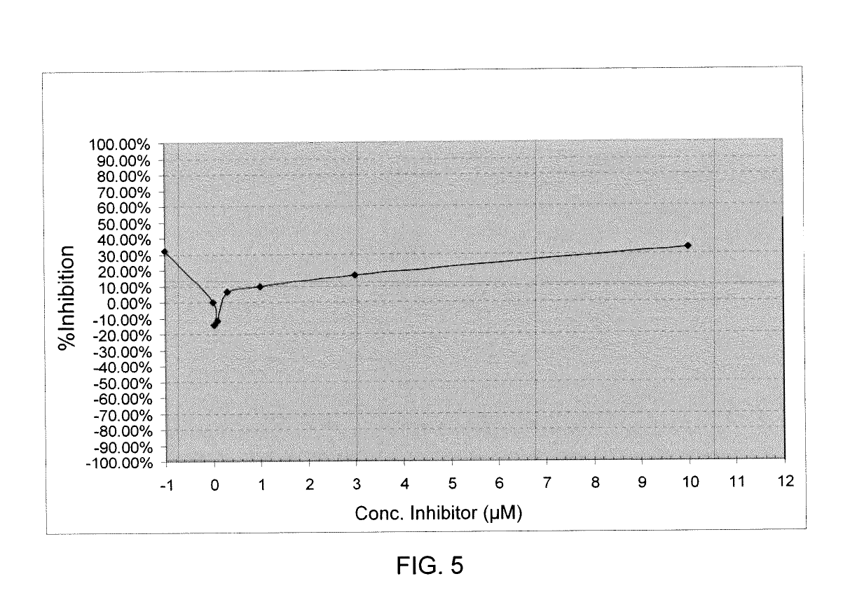

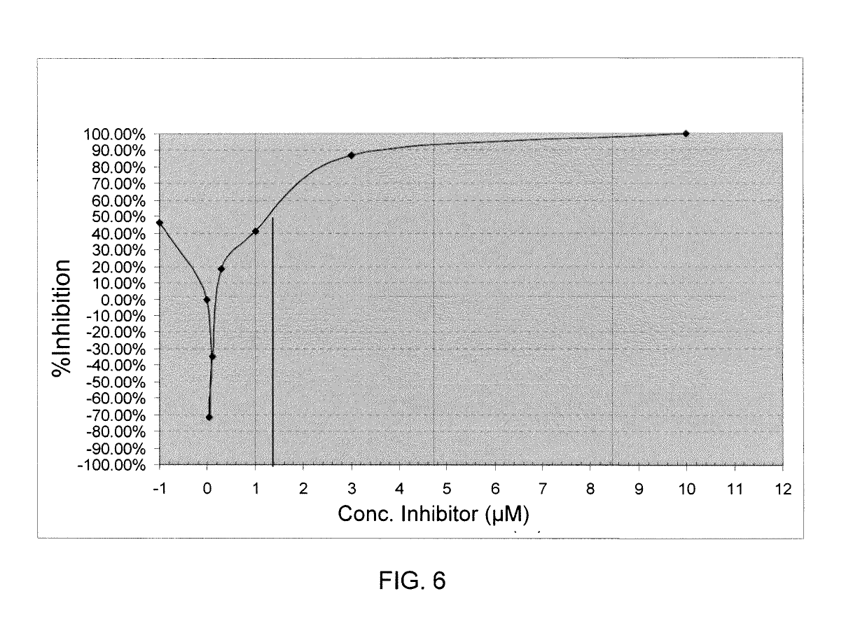

The densitometry showed reduced CTGF protein band intensities, i.e. 37-38 and 42-44 kDa, for the samples at concentrations above 1 μM, indicating diminished protein levels in the samples treated with the ALK-5 inhibitors. The membranes were also probed for the housekeeping gene, glyceraldehydes 3-phosphate dehydrogenase, as an internal standard. As shown in FIGS. 2-6, the half maximal inhibitory concentration (IC50) was calculated to evaluate effectiveness of each inhibitor in inhibiting TGF-β2 function. As the concentration of the inhibitors increased, the percentage TGF-β2 inhibited also increased. One would understand that inhibiting TGF-β2 expression leads to the suppression of ocular scarring following GFS. It is noted that the percentage of inhibition of the growth factor was dependent on the specific concentration of the respective inhibitors administered.

Generally, the growth factor was inhibited to some extent by applying at least 1 μM of inhibitor to the cells. In some cases as much as 3 was required to provide inhibition of the signaling pathway. The control samples prepared without the inhibitors showed no inhibitory function of the TGF-β signaling pathway. It should be noted that in FIGS. 2-6, the “−1” demarcation on the graphs represents the negative expression percentage of the TGF-β downstream protein found when sample 31 was tested and “0” demarcation represents the test data from a sample 32 tested without the respective ALK-5 inhibitor added, but with the TGF-β solution added.

Some samples prepared with low concentrations of the inhibitors actually showed an increase in the activity of the signaling pathway, leading to the conclusion that effective treatment with the inhibitors will be dependent on the specific inhibitor used and the concentration of the inhibitor applied to the surgical site. Moreover, it is desirable to maintain a constant concentration of the inhibitor on the surgical site over a prolonged period of time. Therefore, it may be desirable to apply the inhibitors with methods providing sustained release of the composition, such as with topical gels, polymer implants, and the like.

### Example 2

### Toxicity of ALK-5 Inhibitors In Vitro

A standard of care after a GFS procedure is to treat the surgical site with mitomycin C (MMC) to prevent ocular scarring. MMC, however, is known to cause a high degree of non-selective cell death around the surgical site. This increase in cell death, or high cell toxicity, is known to cause post surgical complications, including an increased rate of late onset post-surgical infections. Therefore, cell toxicity was studied with fibroblasts treated with and without ALK-5 inhibitor SB-505124.

Sample fibroblasts were obtained from New Zealand white rabbit eyes. The fibroblasts were derived from the subconjunctival tissues isolated from the eyes of the subjects. The cells were maintained in 25 cm² flask using 2 ml of medium composed of Eagle's minimal essential medium, 10% fetal bovine serum, 5% calf serum, essential and nonessential aminoacids, and antibiotics. When the cells reached confluence, they were trypsinized and passaged.

Three samples of the fibroblasts were prepared. The first sample, sample A, included fibroblast cultures treated with 2 ng/ml of TGF-β2 for up to 48 hours. The second sample, sample B, included fibroblast cultures that were untreated. The third sample included fibroblast cultures pre-treated with 2 ml of a medium including 10.0 of ALK-5 inhibitor SB-505124 for one hour, and were additionally treated with additional 2 ng/ml of TGF-β2 (R&D Systems, Minneapolis, Minn.) for up to 48 hours.

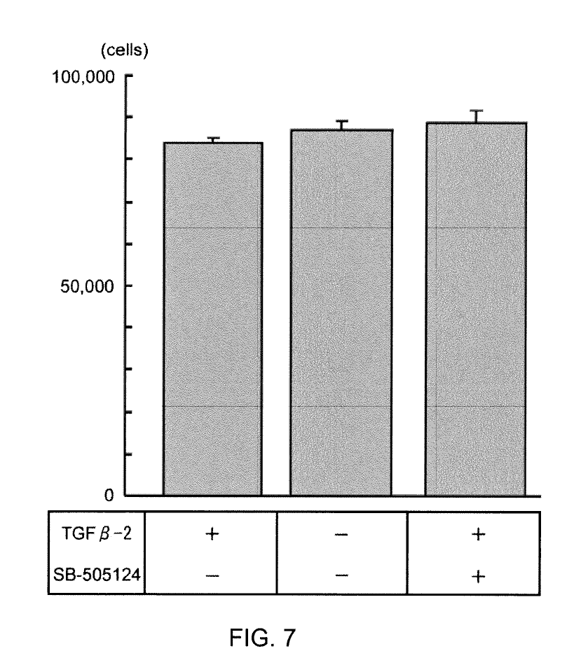

The number of cells were counted using a hemocytometer (Hausser Scientific, Horsham, PC) after trypsinization. As shown in FIG. 7, it was observed that there was no measurable difference in cell number between the sample treated with the ALK-5 inhibitor and that of the samples that were either untreated or treated with TGF-β2 alone. Therefore, in addition to inhibiting the expression of TGF-β2 and mitigating the ocular scarring that may result after GFS by using an ALK-5 type inhibitor, the ALK-5 inhibitor appears to have the added benefit of not destroying healthy cells around the surgical site that prevent post-surgery infection.

### Example 3

### Post Surgical Bleb Survival Results In Vivo

One way to measure the occurrence of ocular scarring following GFS is to measure the failure or survival of the filtration bleb that is created during surgery. As discussed earlier, with reference to FIG. 1, during GFS, instead of draining into the normal drainage site (the trabecular meshwork) 14 of the eye, the aqueous humor is drained into a new “space” that is created under the conjunctiva 12 of the eye. To do this, a small scleral flap in the eye is made. This is followed by the creation of a new drainage route 28 between the opening of the route 20 and a reservoir called a filtration bleb 22. The fluid in the anterior and posterior chamber, called the aqueous humor, can then drain into the bleb 22 via the new drainage route 28 and be absorbed into the vessels around the eye. The bleb 22 and/or the new drainage route 28 can scar and close preventing the aqueous humor from properly draining, called bleb failure.

In order to measure bleb failure and the occurrence of ocular scarring following surgery, four New Zealand white rabbits received GFS and scarring surrounding the surgical sight was monitored. The experimental protocol was approved by the Institutional Animal Care and Use Committee at Northeaster Ohio Universities Colleges of Medicine and Pharmacy. The animal care guidelines are comparable to those published by the US Public Health Service.

The rabbit subjects were anesthetized by subcutaneous injection of a combination of medetomidine, approximately 0.25 to 0.5 mg/kg, and ketamine, approximately 15 to 20 mg/kg. Additional injections, one fourth to one half of the original dose, were also given every 30 to 45 minutes to maintain anesthetization. Local anesthesia was provided with 0.5% proparacaine HCl eye drops.

The surgery was performed under sterile conditions and the eyes were washed with a 1:16 dilution of providone-iodine topical antiseptic and saline. The surgery was performed by retracting the eyelids using a speculum. A partial-thickness corneal traction suture (8-0 silk, Alcon, Fort Worth, Tex.) was placed in the superior cornea to rotate the eye inferiorly. A clear corneal paracentesis tract was made between the 12 and 2 o'clock positions, and Viscoelastic material (0.1-0.2 ml, Discovisc®, Alcon) was injected into the anterior chamber to maintain chamber form.

Surgery was performed at the anterior, temporal and superior sites of the eves. A fornix-based conjunctival flap was raised behind the limbus and a scleral tunnel to the corneal stroma was then fashioned. A 22-G/25-mm venflon 2 cannula (Becton Dickinson, Franklin Lakes, N.J.) was passed through the sclera until it was visible in the cornea. After entry of the cannula into the anterior chamber, the cannula was fixed to the scleral surface with a 10-0 nylon suture (Alcon). The conjunctival incision was closed by interrupted and mattress sutures using 9-0 nylon sutures with a taper cut needle (Ethicon, Somerville, N.J.). One drop of 1% atropine and a single application of combined neomycin and dexamethasone ointment were applied to the ocular surface at the end of surgery.

The first set of subjects, group A, had GFS and was treated with MMC (available from Bedford Laboratories, Bedford, Ohio) as a control (MMC control). Surgical sponges immersed with 0.04% MMC were applied in the subconjunctival space at the surgical site for 5 min right after a fornix-based conjunctival flap was raised. The site was then washed with 500 ml of balanced salt solution.

The second set of subjects, group B, had GFS and were treated with tablets containing 5 mg of SB-505124 and 65 mg lactose (6 mm in diameter, 1.0 mm in thickness), prepared using a compression technique. Prior to sutures of the conjunctival incision during GFS, the lactose tablet was broken into several pieces and placed on the sclera at the surgical site.

The third and fourth set of subjects, groups C and D, were given GFS without any treatment (no adjunct control) and GFS with lactose tablets devoid of inhibitors (tablet control), respectively.

After GFS, all of the animals in groups A-D were examined under a slit-lamp daily during the first week after GFS, and at least twice a week thereafter until the time bleb failure was observed, or up to 28 days post surgery, whichever was longer. The filtering bleb, anterior chamber activity and depth, conjunctival hyperemia and aqueous humor leakage were examined. Bleb failure was defined as the appearance of a flat, vascularized, scarred bleb in association with a deep anterior chamber. The lack of aqueous humor drainage into the bleb promoted by eye massage assisted in the judgment of bleb failure. Filtering blebs were also photographed with a digital camera (FinePix F40fd, Fujifilm, Japan).

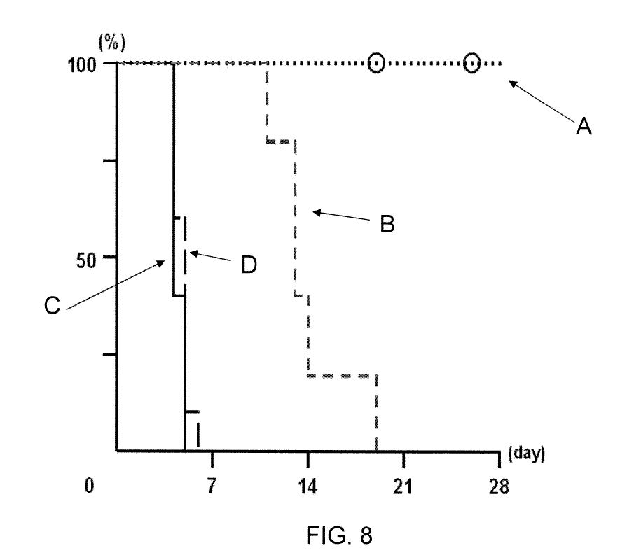

As shown in FIG. 8, the subjects in Groups A and B, treated with MMC and the ALK-5 inhibitor, respectively, had filtering blebs that were maintained for at least 10 days after GFS, while the filtering blebs in the subjects of groups C and D, that received no control treatment or a lactose tablet control, failed within one week after surgery. This indicates that like the MMC, the ALK-5 inhibitor was able to prevent ocular scarring following surgery.

### Example 4

### Post Surgery IOP Results In Vivo

IOP for the animals in groups A-D was measured with a TONO-PEN AVIA® (Reichert Ophthalmic Instruments, Depew, N.Y.) by gently touching the cornea of each subject after topical anesthesia was applied with 0.5% proparacaine. The IOP reading was omitted if its confidence interval was less than 95%. An average of three measurements was taken to deduce IOP.

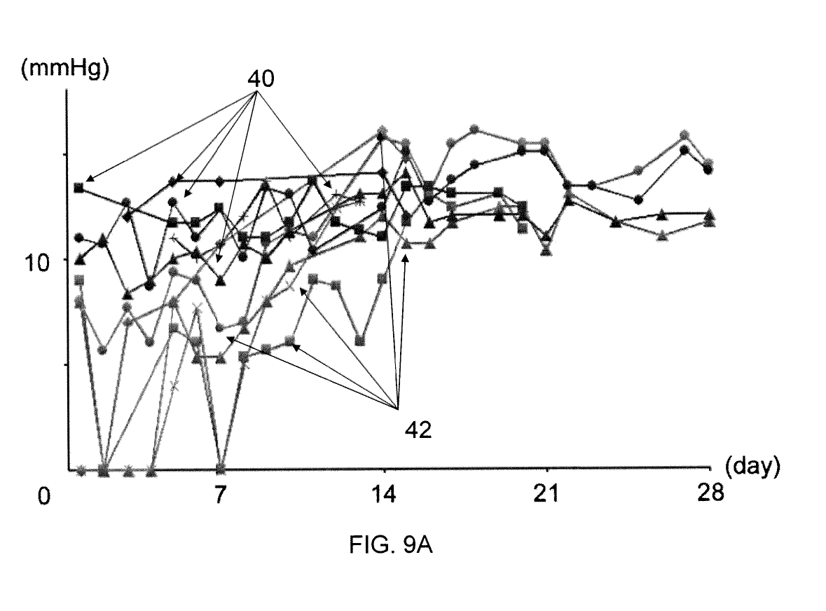

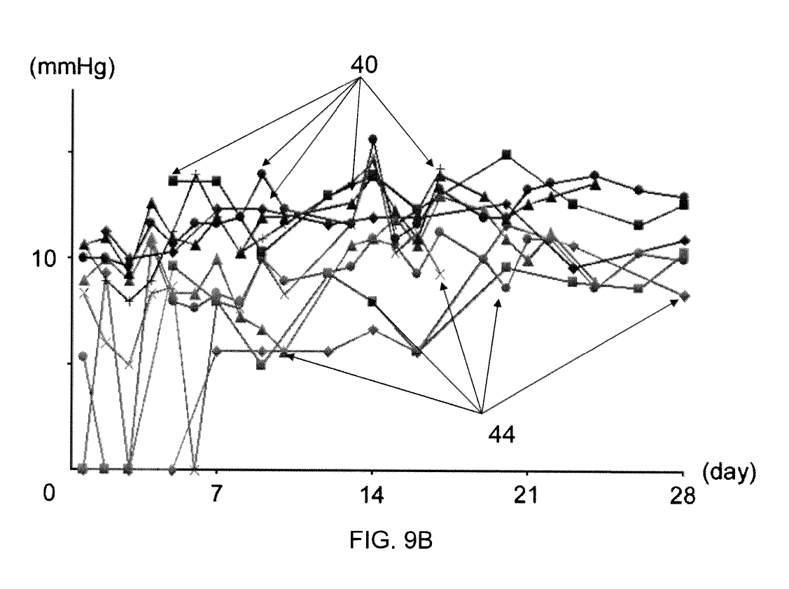

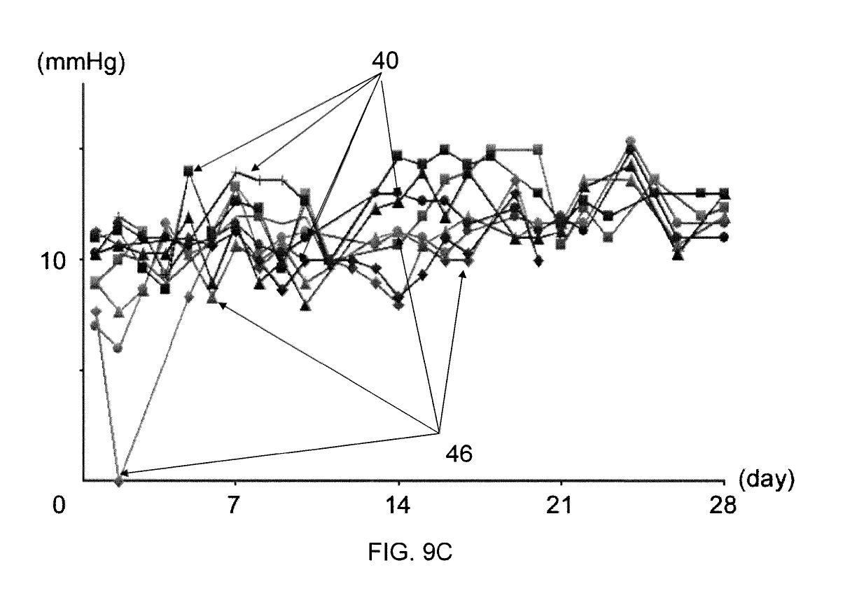

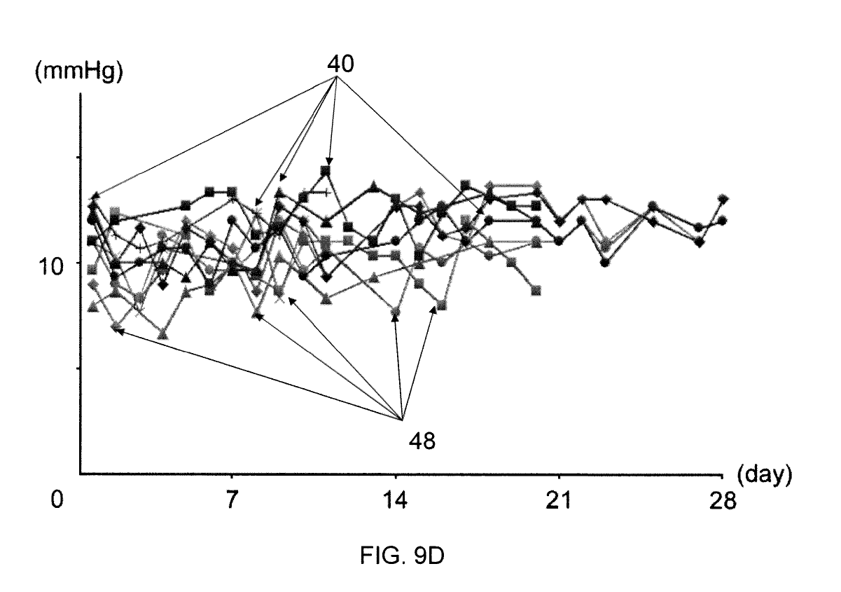

FIG. 9A shows the comparison of the IOP of an untreated eye 40, compared to that of the surgical subjects treated with SB-505124 inhibitor 42. FIG. 9B shows the comparison of the IOP of an untreated eye 40, compared to that of the surgical subjects treated with an MMC control 44. FIG. 9C shows the comparison of the IOP of an untreated eye 40, compared to that of the surgical subjects treated without any applications (no adjunct control) 46. FIG. 9D shows the comparison of the IOP of an untreated eye 40, compared to that of the surgical subjects treated with the tablet of 100% lactose (tablet control). As shown in FIGS. 9A-D, as with the non-surgically treated eyes, the IOP of treated eyes was lowered for up to 10 days for the group treated with SB-505124 inhibitor, and throughout the observation period for the treated with MMC. The difference in the IOP in groups of lactose control or no adjunct control was slight, leading to the conclusion that the use of the inhibitor, like the MMC, leads to decreased IOP following surgery.

### Example 5

### Hemotoxylin and Eosin Staining Results Ex Vivo

Five days after surgery, the subjects in Groups A-D were euthanized with Fatal-plus™ (Vortech Pharmaceutical, Dearborn, Mich.) following the manufacturer's instructions. The subject's eyes were enucleated with palpebral margins to keep the conjunctiva epithelium and the subconjunctival space intact. Enucleated eyes were fixed with 10% buffered formalin and 5 μm thick paraffin sections were prepared. The sections were stained with hematoxylin and eosin (H&E) for histological examination, and images were captured using an Olympus DX51 light microscope and DP controller (Olympus, Tokyo, Japan).

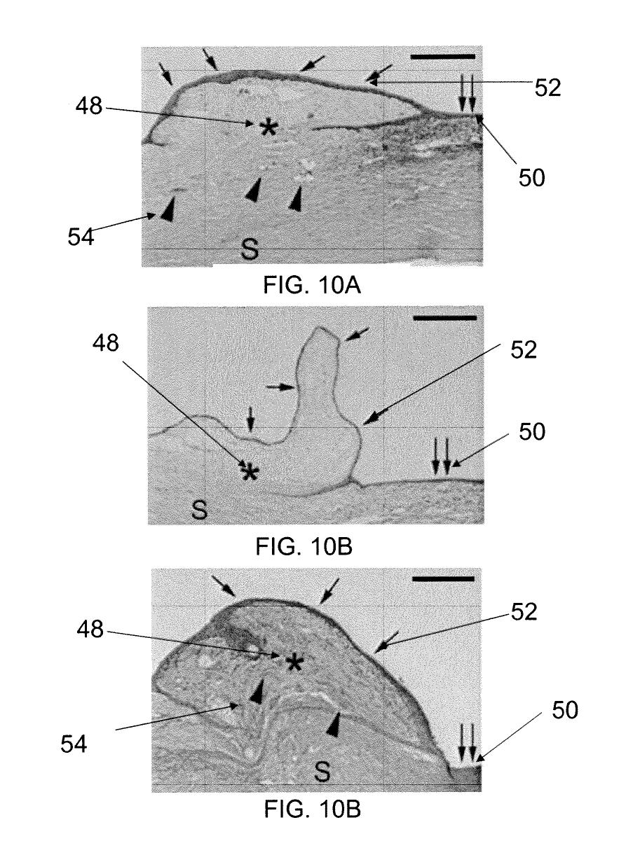

FIG. 10 is images of Hematoxylin and Eosin staining in tissue sections from eyes five days after surgery with SB-505124 (10 A) and MMC (MMC control, 10 B) and without any adjunct (no adjunct controlm 10 C). Infiltration of only a few inflammatory cells and mild scarring were observed in the subconjunctival space 48 of eyes in the GFS with SB-505124 (10 A) or the MMC control (10 B), whereas numerous inflammatory cells and massive scarring were seen in the no adjunct control (10 C). At the corneal limbus 50, infiltration of cells was observed in all. Thinner conjunctival epithelium 52 was seen in the MMC control (10 B) compared to that in the GFS with SB-505124 (10 A) and no adjunct control (10 C). Subconjunctival blood vessels 54 were seen in GFS with SB-505124 (10A) and no adjunct control (10 C) in general, but seldom in the MMC control (10 B). Thus, like the untreated subjects, these images demonstrate that the subjects treated with SB-505124 inhibitor show no or very low signs of tissue toxicity. Therefore, one can conclude that the mechanism in suppression of ocular scarring by SB-505124 is different from that of MMC, which suppresses ocular scarring due to wide spread cell death.

### Example 5

### Toxicity of ALK-5 Inhibitors by Measuring Cell Outgrowth Ex Vivo

To further investigate if the ALK-5 inhibitor's ability to suppress ocular scarring was related to its toxicity, like MMC, a subconjunctival tissue fibroblast outgrowth assay was performed. Five days after GFS, subjects prepared as in Groups A and B, with MMC or the ALK-5 inhibitor, were euthanized as described above. Under an ophthalmic surgical microscope, subconjunctival tissues were dissected from the surgical site and 180 (6-o'clock position) from the surgical site (the 180° control). Each biopsy specimen (explant) was placed in a 25 cm² cell culture flask with complete media. Care was taken in handling the biopsy specimens, in particular to ensure that the samples did not dry out and affect the cellular viability. The cell outgrowth, in mm, from the edge of each explant was observed, and the length of the outgrowth in 4 quadrants was measured.

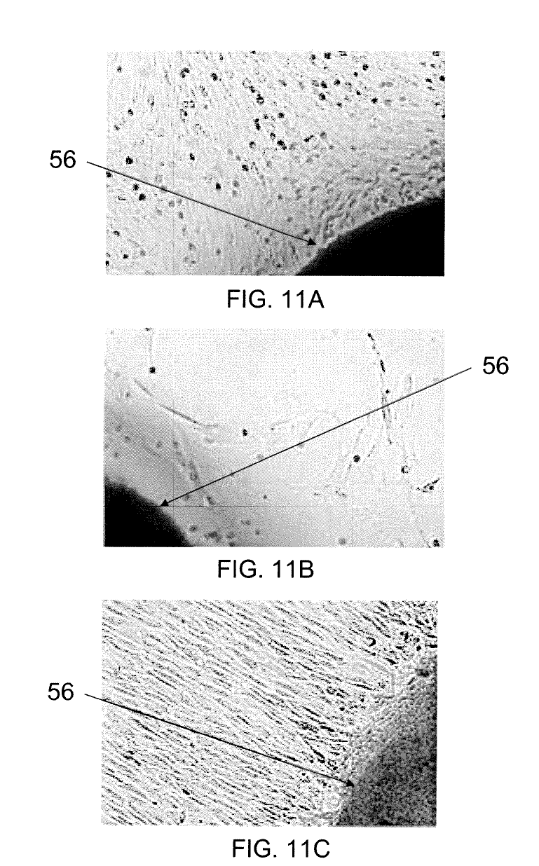

FIGS. 11A-C are images of cell outgrowth from explants taken from the subjects treated with SS-505124. MMC, and with no control, respectively. As shown in FIG. 11A, cell outgrowth 56 from the edge of the subconjunctival tissue explant for the subject treated with SB-505124 was robust, while the cell outgrowth 56 was poor for those treated with MMC. As shown in FIGS. 11A and 11C, there was no significant difference in cell outgrowth between subconjunctival tissues treated with SB-505124 and the subjects in the untreated group, whereas, as shown in FIGS. 11B and 11C, a significant difference was noted between the MMC group and the untreated control group subjects. A few small, round specks that appear to be dead cells were noted on top of the outgrowth from tissues treated with the ALK-5 inhibitor and MMC, but seldom in that from the untreated control group.

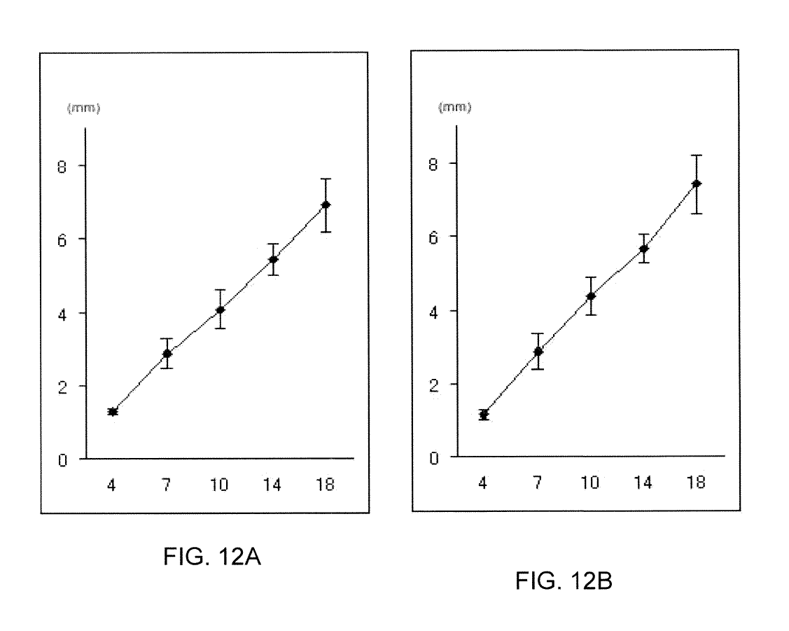

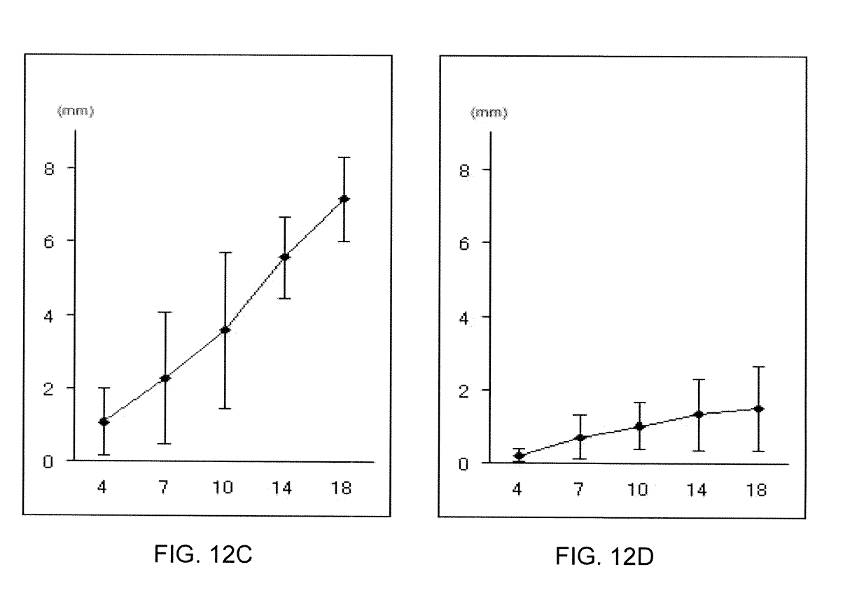

FIGS. 12A-D are the graphical representations of the cell outgrowth length measured from the explants taken from the SB-505124 180° control, the MMC 180 control, SB-505124 surgical site, and the MMC surgical site, respectively. There appeared to be no significant difference in the amount of cell outgrowth measured for the explants shown in Figs. A-C, approximately 7 mm over 18 days. However, the cell outgrowth measured from the explant taken from the surgical site of the tissue treated with MMC was very small, less than 2 mm over 18 days. Therefore, it appears that, like untreated tissue, tissue treated with SB-505124 does not cause significant cell toxicity, unlike MMC.

In order to evaluate the statistical significance of the results, a two-way ANOVA was performed to evaluate the IOP changes after GFS and the fibroblast outgrowth from subconjunctival tissues. A Kaplan-Meier method was used to analyze the bleb survival (SPSS ver.16, Chicago, Ill.).

One of skill in the art would recognize that the examples and methods disclosed herein are intended to be exemplary in nature and in no way are intended to limit the claimed invention.

What is claimed is:

1. A method for suppressing ocular scaring following an ocular surgical procedure comprising:
providing a composition comprising an effective amount of an ALK-5 inhibitor, in an amount sufficient to inhibit the signaling pathway of TGF-β, and a pharmaceutically acceptable vehicle therefore, wherein the ALK-5 inhibitor is selected from the group consisting of: 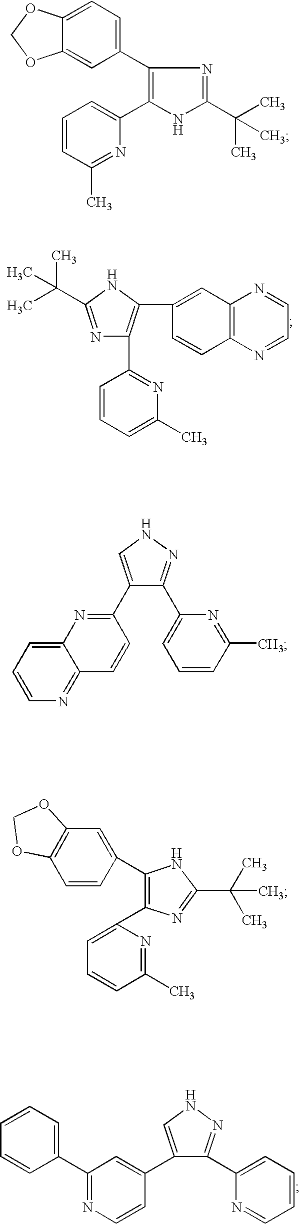 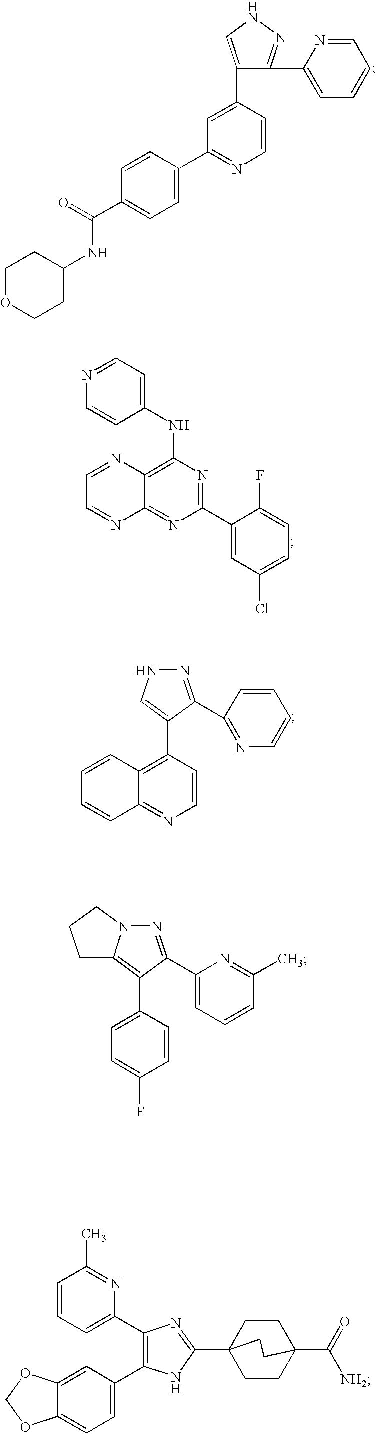 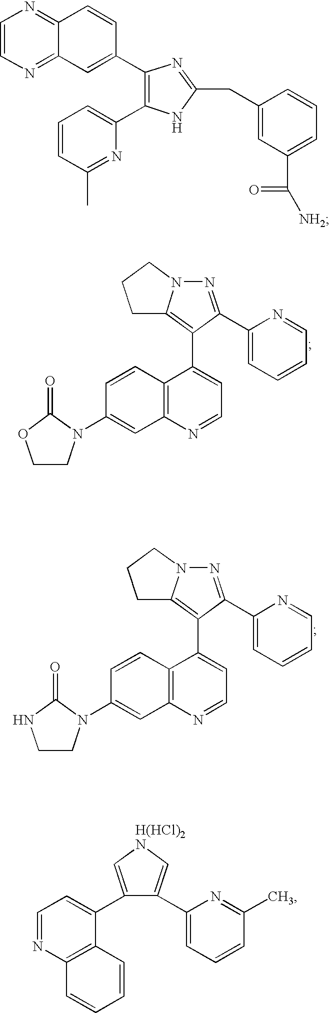
and combinations thereof;
wherein application of the composition to a patient's eye suppresses the formation of scar tissue following the ocular surgical procedure.

2. The method of claim 1, wherein the ALK-5 inhibitor is a pharmaceutically acceptable salt thereof.

3. The method of claim 1, wherein the composition is applied in the form of a topical application to a post-surgical surgical site following the ocular surgical procedure.

4. The method of claim 1, wherein the pharmaceutically acceptable vehicle includes a sustained release polymer carrier.

5. The method of claim 1, wherein the pharmaceutically acceptable vehicle further includes a carrier medium capable of forming a gel upon administration to a surgical site on the patient's eye.

6. The method of claim 1, wherein the pharmaceutically acceptable vehicle further includes a carrier medium in the form of a tablet.

7. The method of claim 1, wherein the ALK-5 inhibitor is present in the composition in an amount from about 1.0 to about 30.0 μM.

8. The method of claim 1, wherein the ALK-5 inhibitor is selected from the group consisting of: 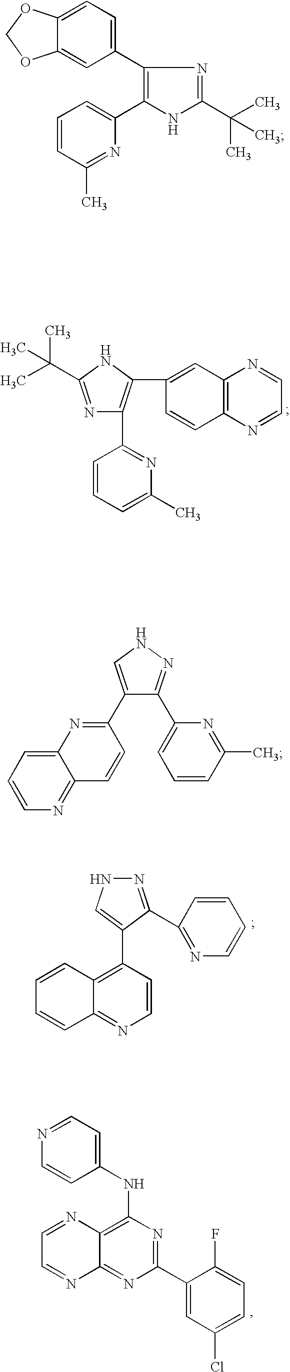
and combinations thereof.

9. The method of claim 8, wherein the ALK-5 inhibitor is a pharmaceutically acceptable salt thereof.

10. The method of claim 8, wherein the composition is applied in the form of a topical application to a post-surgical surgical site following the ocular surgical procedure.

11. The method of claim 8, wherein the pharmaceutically acceptable vehicle includes a sustained release polymer carrier.

12. The method of claim 8, wherein the pharmaceutically acceptable vehicle further includes a carrier medium capable of forming a gel upon administration to a surgical site on the patient's eye.

13. The method of claim 8, wherein the pharmaceutically acceptable vehicle further includes a carrier medium in the form of a tablet.

14. The method of claim 8, wherein the ALK-5 inhibitor is present in the composition in an amount from about 1.0 to about 30.0 μM.

15. The method of claim 8, wherein the ALK-5 inhibitor comprises:

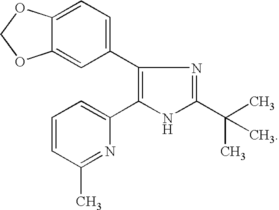

16. The method of claim 15, wherein the ALK-5 inhibitor is a pharmaceutically acceptable salt thereof.

17. The method of claim 15, wherein the composition is applied in the form of a topical application to the post-surgical surgical site following the ocular surgical procedure.

18. The method of claim 15, wherein the pharmaceutically acceptable vehicle includes a sustained release polymer carrier.

19. The method of claim 15, wherein the pharmaceutically acceptable vehicle further includes a carrier medium capable of forming a gel upon administration to a surgical site on a patient's eye.

20. The method of claim 15, wherein the ALK-5 inhibitor is present in the composition in an amount from about 1.0 to about 30.0 μM.
